(19) United States
(12) Patent Application Publication
(10) Pub. No.: US 2025/0000827 A1
(43) Pub. Date: Jan. 2, 2025

(54) TOPICAL COMPOSITION FOR PAIN RELIEF

(71) Applicant: Johnson & Johnson Consumer Inc., Skillman, NJ (US)

(72) Inventors: Eleanor F. Small, Philadelphia, PA (US); Edward Crudden, Jenkintown, PA (US); Austin Courtney Suder, Downingtown, PA (US); Mackenzie Hannum, Philadelphia, PA (US)

(21) Appl. No.: 18/745,050

(22) Filed: Jun. 17, 2024

Related U.S. Application Data
(60) Provisional application No. 63/510,760, filed on Jun. 28, 2023.

(51) Int. Cl. A61K 31/167; A61K 9/00; A61K 31/045; A61K 31/125; A61P 29/00

(52) U.S. Cl. CPC A61K 31/167; A61K 9/0014; A61K 31/045; A61K 31/125; A61P 29/00

(57) ABSTRACT

The present invention provides a topical composition including a topical analgesic, a viscosity increasing agent, a humectant, an emollient, and an emulsifier. The topical composition is substantially free of volatile alcohol. Additionally, the present invention includes methods of relieving pain. The methods include topically applying to an exterior skin portion of the user's body in need of pain treatment a composition including a topical analgesic, a viscosity increasing agent, a humectant, an emollient, and an emulsifier.

### CROSS REFERENCE TO RELATED APPLICATIONS

This application claims the benefit of U.S. provision application 63/510,760 filed on Jun. 28, 2023, the complete disclosure of which is hereby incorporated herein by reference for all purposes.

### FIELD OF THE INVENTION

The present invention relates to topical compositions for pain relief; in particular, for pain relief of users with varying skin sensitivities and/or preferences for sensations on the skin that may indicate pain relief. More specifically, the present invention relates to a topical composition including a topical analgesic, as well as at least one viscosity increasing agent, at least one humectant, at least one emollient and at least one emulsifier, where the composition is substantially free of volatile alcohol.

Additionally, the present invention relates to methods of relieving pain, especially in users with sensitive skin and/or in users who prefer certain sensations for pain relief. In particular, such methods include topically applying to an exterior skin portion of a user's body a composition including a topical analgesic, as well as at least one viscosity increasing agent, at least one humectant, at least one emollient and at least one emulsifier, where the composition is substantially free of volatile alcohol.

### BACKGROUND OF THE INVENTION

Pain is a common experience that may be of short duration (called “acute pain”) or of longer duration, for example, six months or longer (called “chronic pain”). Pain may negatively impact a person's quality of life and treatment of pain may be costly. Pain treatment options that are effective and inexpensive are desirable.

Body pain is often treated with a topical pain cream. Topical pain creams currently exist in various forms on the consumer market. However, such creams often provide a harsh user experience causing dry, red, irritated skin, an overly strong cooling or warming sensation and/or an overwhelming scent. Such creams are unsuitable for consumers with sensitive skin.

“Sensitive skin” is defined as the skin of any part of the body that has discomfort at any given time. Examples of conditions causing sensitive skin may include, but are not limited to, acne, contact dermatitis, dry skin, itchy skin and eczema. Sensitive skin may be described as skin that is “reactive” to external stimuli or that presents a special need. Examples of external stimuli may include sunlight, wind, water, air temperature, air moisture, skin and body hydration, diet and/or contact with chemical or physical materials.

Consumers with sensitive skin often find it difficult to find topical cosmetic or medicinal products that do not irritate or further irritate their skin. This is particularly detrimental to sensitive skin consumers who have health or cosmetic concerns (i.e., concerns unrelated to their sensitive skin) that are commonly addressed by topical skin treatments. One such example may be sensitive skin consumers who also suffer from body pain, for example pain in the neck, forearm, elbow, upper arm, wrist, calf, thigh, knee, back, shoulder, hip and/or ankle.

A “sensate” in a topical cosmetic or medicinal product is defined as an ingredient that imparts a sensation to the user upon contact. The sensation may start immediately upon contact with the sensate or the sensation may be delayed for a certain period of time. The sensation may be, for example, a warming or a cooling sensation.

Sensates in topical products may cause skin sensitivity. Patients that are sensitive to warming agents have sensitivity to warming, described as too intense, too hot or contributing a burning sensation. Likewise, some patients that are sensitive to cooling agents have sensitivity to cooling, described as too intense, too cold or contributing to numbness of the skin. When a consumer or patient experiences skin sensitivity or a negative sensation while using a topical product, that consumer or patient will be unlikely or reluctant to try that topical product again regardless of whether the product was effective in pain relief.

Additionally, not all consumers and patients are alike. Cooling and warming perception of a patient correlates to the interaction of the topical product with the patient's nerves and the number of nerves in the patient's skin. A square inch of human skin has approximately 1,300 pain receptors, where approximately 40 pain receptors per square inch are for cold and approximately six pain receptors per square inch are for warmth. Based on differences in the skin, each patient and consumer may experience skin sensitivity and sensation differently. Therefore, a topical pain product may cause skin sensitivity to one person, but not to another. Likewise, a topical pain product with a sensate may cause a positive sensation, a negative sensation or no sensation depending on the user.

As such, there exists a need for topical pain compositions that provide different benefits to users depending on user preference. For example, there exists a need for a topical pain composition that provides a milder experience for users or consumers with sensitive skin. There exists a need for a topical pain composition that provides a mild warming sensation or the absence of a warming sensation for users that have sensitivity to warming. There likewise exists a need for a topical pain composition that provides a mild cooling sensation or the absence of a cooling sensation for users that have sensitivity to cooling. Additionally, there exists a need for topical pain compositions that provide benefits such as being easy to apply, have a mess free application, are non-sticky, are non-greasy, and do not leave a residue. Such topical compositions could be used, for example as daily treatment, leave-on products.

### SUMMARY OF THE INVENTION

According to an example, a topical composition for pain relief may include a topical analgesic, at least one viscosity increasing agent, at least one humectant, at least one emollient and at least one emulsifier, where the topical composition is substantially free of volatile alcohol. Such topical composition may also be suitable for users with sensitive skin.

According to an example, the topical composition may include a topical analgesic, a viscosity increasing agent in an amount of from about 0.05 to about 1.2 wt %, a humectant in an amount of from about 1.0 to about 5.0 wt %, an emollient in an amount of from about 2.5 to about 5.0 wt %, and an emulsifier in an amount of from about 0.5 to about 2.0 wt %, where the topical composition is substantially free of volatile alcohol.

In one example, the topical analgesic may be in an amount of about 0.5% to about 10% by weight. The topical analgesic may be one or more of lidocaine, camphor, menthol or benzocaine.

In an example, the topical analgesic may be lidocaine. The lidocaine may be in an amount of about 4% by weight.

In another example, the topical analgesic may be camphor and menthol. The camphor may be in an amount of about 4% by weight and the menthol may be in an amount of about 1.25% by weight.

In one example, the topical composition may further include a cooling agent. The cooling agent may be menthol or menthol lactate.

In another example, the topical composition may further include a warming agent. The warming agent may be camphor, vanilla butyl ether or other vanilloid. The warming agent may be vanilla butyl ether or other vanilloid may be in an amount of about 0.25 to about 0.5% by weight.

In an example, the topical composition may include both a warming agent and a cooling agent. The warming agent may be one or more of camphor, vanilla butyl ether or other vanilloid. The cooling agent may be menthol or menthol lactate. In one example, the warming agent may be camphor and the cooling agent may be menthol. For example, the camphor and the menthol may be in a weight ratio of 3.2:1. In another example, the warming agent may further include vanilla butyl ether.

In an example, wherein the topical composition may be free of a cooling agent or a warming agent. In other various examples, the topical composition may be free of lidocaine and/or menthol and/or camphor and/or methyl salicylate and/or capsaicin.

In various examples, the viscosity increasing agent may be in an amount of about 0.6% by weight. The humectant may be in an amount of about 3.0% by weight. The at least one emollient may be in an amount of about 3.5% by weight. The emulsifier may be in an amount of about 1.0% by weight.

Another example contemplates a method of relieving pain in a user with sensitive skin, by applying a one or more of the topical compositions contemplated herein to an exterior skin portion of the user's body in need of pain treatment. The exterior skin portion of the user's body may include at least one of a user's neck, back, legs, hands, fingers and wrists.

### DETAILED DESCRIPTION

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination. Unless otherwise defined, all technical and scientific terms used herein have the same meaning as commonly understood by one of ordinary skill in the art to which this disclosure pertains. The terminology used in the description is for describing particular examples only and is not intended to be limiting of the disclosure.

In the disclosure, the singular forms “a,” “an,” and “the” include the plural reference, and reference to a particular numerical value includes at least that particular value, unless the context clearly indicates otherwise. Thus, e.g., a reference to “a material” is a reference to at least one of such materials and equivalents thereof known to those skilled in the art, and so forth.

When a value is expressed as an approximation by use of the descriptor “about” it will be understood that the particular value forms another example. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. In some cases, the descriptor “about” will be understood as meaning ±up to 10%, for example ±10, 9, 8, 7, 6, 5, 4, 3, 2 or 1%. Where present, all ranges are inclusive and combinable. That is, references to values stated in ranges include every value within that range.

When a list is presented, unless stated otherwise, it is to be understood that each individual element of that list and every combination of that list is to be interpreted as a separate example. For example, a list of examples presented as “A, B, or C” is to be interpreted as including the examples, “A,” “B,” “C,” “A or B,” “A or C,” “B or C,” or “A, B, or C.”

It is to be appreciated that certain features of the invention which are, for clarity, described herein in the context of separate examples, may also be provided in combination in a single example. That is, unless obviously incompatible or excluded, each individual example is deemed to be combinable with any other example(s) and such a combination is considered to be another example. Conversely, various features of the invention that are, for brevity, described in the context of a single example, may also be provided separately or in any sub-combination. It is further noted that the claims may be drafted to exclude an optional element. As such, this statement is intended to serve as antecedent basis for use of such exclusive terminology as “only” and the like in connection with the recitation of claim elements, or use of a “negative” limitation. Finally, while an example may be described as part of a series of steps or part of a more general structure, each said step may also be considered an independent example in itself.

It is believed that one skilled in the art can, based on the description herein, utilize the present invention to its fullest extent. The following specific examples are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% wt,” “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a “product” is optionally in finished packaged form. In one example, the package is a container such as a plastic, metal or glass tube or jar containing the composition. In one example, the composition may be squeezed or pumped out of the container. In another example, the composition may be applied to the skin using an applicator, such as a roller, roller ball(s), brush, a massage element or other functional applicator. According to a further example, the composition may be suitable or may not be suitable for a spray or aerosol dispensing system. The product may further contain additional packaging such as a plastic or cardboard box for storing such container. In one example, the product includes a composition of the invention and contains instructions directing the user to apply the composition to the skin.

As used herein, “topically applying” means directly laying on or spreading on outer skin, by use of the hands or an applicator such as a wipe, roller, or spray.

As used herein, “topical composition” means a composition that is applied to the outer skin of a user.

As used herein, “pain relief” refers to the alleviation of pain at one or more locations of the body.

As used herein, “cosmetically acceptable” means that the ingredients the term describes are suitable for use in contact with tissues (e.g., the skin or hair) without undue toxicity, incompatibility, instability, irritation, allergic response, or the like.

As used herein, the term “safe and effective amount” means an amount sufficient to induce the desired effect, but low enough to avoid serious side effects. The safe and effective amount of the compound, extract, or composition will vary with, e.g., the age, health and environmental exposure of the end user, the duration and nature of the treatment, the specific extract, ingredient, or composition employed, the particular carrier utilized, and like factors.

One aspect of the invention pertains to a topical composition for pain relief that is suitable for users or consumers with sensitive skin. The topical composition includes a topical analgesic, as well as at least one viscosity increasing agent, at least one humectant, at least one emollient and at least one emulsifier, where the composition is substantially free of volatile alcohol.

As used herein, “analgesic” means a class of drugs designed to relieve pain without causing the loss of consciousness. Such analgesics may be over-the-counter (OTC) or prescription drugs. Examples of analgesics may include, for example, acetaminophen, aspirin, COX inhibitors, and nonsteroidal anti-inflammatory drugs (NSAIDs) and salts and derivatives thereof.

A “topical analgesic” refers to a drug, medication or pain-relieving agent that is applied on the skin to relieve muscle, joint or nerve pain. Topical analgesics may also be classified as counterirritants or anti-inflammatory agents in certain cases. Topical analgesics may be absorbed by the skin and may act on the tissue beneath. Examples of suitable topical analgesics may include lidocaine, capsaicin, nonsteroidal anti-inflammatory drugs (NSAIDs), salicylate rubefacients, benzocaine, tetracaine, nitroglycerin, rubefacients, such as camphor, menthol, methyl nicotinate and methyl salicylate, and salicylates, such as aspirin, magnesium salicylate and sodium salicylate and salts and derivatives thereof. Capsaicin, menthol, methyl salicylate and camphor, for example, may also be classified as counter-irritants. And, in certain examples, as discussed below, menthol may be further described as a cooling agent or cooling sensate, and camphor may be further described as a warming agent or warming sensate. A topical analgesic may include a combination of those listed above.

According to an example, the analgesic or topical analgesic may be recommended for external application to the skin, also referred to as the “exterior skin portion” of a user's body. For example, the topical analgesic should not be applied to internal body surfaces, such as the oral cavity. For this reason, the term “external analgesic” may be used simultaneously or in lieu of “topical analgesic.”

The analgesic or topical analgesic may be present in amounts ranging from about 0.1, 0.5, 1, 1.5, 2, 2.5, 3.1, 3.2, 3.3, 3.4, 3.5, 3.6, 3.7, 3.8, 3.9, 4, 4.5, or 5 to about 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 10 wt. % of the total composition. In one or more examples, the analgesic or topical analgesic is present in an amount ranging from about 0.1 wt. % to about 10 wt. % by total weight of the composition. In some examples, the analgesic or topical analgesic is present in an amount ranging from about 0.5 wt. % to about 8 wt. % by total weight of the composition. In one or more examples, the analgesic or topical analgesic is present in an amount ranging from about 2 wt. % to about 6 wt. % by total weight of the composition. In some examples, the analgesic or topical analgesic is present in an amount ranging from about 3 wt. % to about 5 wt. % by total weight of the composition. In one or more examples, the analgesic or topical analgesic is present in an amount ranging from about 3.5 wt. % to about 4.5 wt. % by total weight of the composition. In some examples, the analgesic or topical analgesic is present in an amount of about 4 wt. % by total weight of the composition.

In one or more examples, the topical analgesic may comprise lidocaine, and is present in an amount ranging from about 0.1 wt. % to about 10 wt. % by total weight of the composition. In some examples, the lidocaine is present in an amount ranging from about 0.5 wt. % to about 8 wt. % by total weight of the composition. In one or more examples, the lidocaine is present in an amount ranging from about 2 wt. % to about 6 wt. % by total weight of the composition. In some examples, the lidocaine is present in an amount ranging from about 3 wt. % to about 5 wt. % by total weight of the composition. In one or more examples, the lidocaine is present in an amount ranging from about 3.5 wt. % to about 4.5 wt. % by total weight of the composition. In some examples, the lidocaine is present in an amount of about 4 wt. % by total weight of the composition. Lidocaine may be present in a salt form such as lidocaine hydrochloride.

In an example, the topical analgesic may comprise menthol. The menthol may be present in an amount of about 0.1 wt. % to about 5 wt. % by total weight of the composition, for example about 0.1% wt, about 0.2% wt, about 0.3% wt, about 0.4% wt, about 0.5% wt, about 0.6% wt, about 0.7% wt, about 0.8% wt, about 0.9% wt, about 1.0% wt, about 1.1% wt, about 1.2% wt, about 1.25% wt, about 1.3% wt, about 1.4% wt, about 1.5% wt, about 1.6% wt, about 1.7% wt, about 1.8% wt, about 1.9% wt, about 2.0% wt, about 2.1% wt, about 2.2% wt, about 2.3% wt, about 2.4% wt, or about 2.5% wt. In one example, the menthol may be about 1.25% by weight.

In an example, the topical analgesic may comprise camphor. The camphor may be present in an amount of from about 0.1 wt. % to about 10 wt. %, or from about 0.5 wt % to about 8 wt % by total weight of the composition. For example, camphor may be present in an amount of about 0.1 wt %, about 0.5% wt, about 1.0% wt, about 1.5% wt, about 2.0% wt, about 2.5% wt, about 3.0% wt, about 4.0% wt, about 4.5% wt, about 5.0% wt, about 5.5% wt, about 6.0% wt, about 6.5% wt, about 7.0% wt, about 7.0% wt, about 8.0% wt, about 8.5% wt, about 9.0% wt, about 9.5% wt, or about 10.0% wt. In one example, the camphor may be about 4.0% by weight.

In another example, the topical analgesic may comprise a combination of camphor and menthol. For example, the combination may include camphor in an amount of about 4.0% by weight and menthol in an amount of about 1.25% by weight.

According to an example, the composition may also exclude certain ingredients. The term “substantially free of” refers to a composition having less than 5% by weight, less than 4% by weight, less than 3% by weight, less than 2% by weight, less than 1% by weight, less than 0.75% by weight, or less than 0.5% by weight of a particular ingredient. The term “completely free of” refers to a composition having less than 0.5% by weight, less than 0.4% by weight, less than 0.3% by weight, less than 0.2% by weight, less than 0.1% by weight or the complete absence of an ingredient.

In one example, the composition may be substantially free or completely free of certain topical analgesics, for example lidocaine, menthol and/or camphor.

According to an example, the composition may be substantially free of volatile alcohol. The term “volatile alcohol” refers to an alcohol which will readily vaporize, or an alcohol with a vapor pressure which is higher than that of water at 20° C. Examples of volatile alcohols include, but are not limited to, specially denatured (SD) alcohol, for example SD Alcohol 40, denatured alcohol, ethanol, methanol and isopropyl alcohol. The vapor pressure of various alcohols at 20° C. include ethanol with a vapor pressure of 5.95 kPa (kilopascals), methanol with a vapor pressure of 11.9 kPa and isopropanol with a vapor pressure of 4.40 kPa.

According to an example, the composition may be substantially free of or completely free of cooling agents such as menthol, or cooling sensates such as menthyl esters like menthol lactate. Other examples of cooling agents may include menthyl glutarate, N-ethyl 2-isopropyl-5-methylcyclohexanecarboxamide, N-[(ethoxycarbonyl)methyl)-p-menthane-3-carboxamide and (1R,2S,5R)-N-(4-methoxyphenyl)-5-methyl-2-(1-methylethyl) cyclohexanecarboxamide. Cooling agents may include a phenyl ring and a polar side group, for example Menthyl Glutarate. In certain examples a combination of more than one cooler comprising a phenyl ring with a polar side group may be used as a cooling agent. In one example a combination of a cooler comprising a phenyl ring with a polar side group and a cooler without a phenyl ring with a polar side group may be used as a cooling agent. In another example, the cooler comprising a phenyl ring with a polar side group is a cooler with a cyclohexyl core substituted at ring positions 1,3 and 4 and an ester or an amide group at one of the three positions.

According to an alternative example, the topical composition may contain a cooling agent such as menthol, or cooling sensates such as menthyl esters like menthol lactate. The cooling agent may be menthol laevo pellets. In this example the composition may contain from about 0.1 to about 10% by weight, or about 0.5 to about 3, or about 0.5 to about 2 weight % of at least one cooling agent. In an example, the composition contains about 0.5% wt, about 0.6% wt, about 0.7% wt, about 0.8% wt, about 0.9% wt, about 1.1% wt, about 1.2% wt, about 1.3% wt, about 1.4% wt, or about 1.5% wt. In an example the composition contains about 1% by weight of at least one cooling agent.

According to an example, the composition may be substantially free of or completely free of warming agents such as vanilla butyl ether, camphor, methyl salicylate, capsaicin or ginger extract. According to an example, the composition may contain one or more warming agents such as vanilla butyl ether and/or other vanilloids, capsaicin and/or other capsaicinoids, ginger extract, camphor and/or methyl salicylate. According to another example, the composition may contain vanilla butyl ether, but be substantially free of or completely free of capsaicin and other warming agents. The vanilla butyl either (“VBE”), for example, may be that sold under the tradename “HOTACT®” sold by Takasago Corporation.

In an alternative example the composition may contain from about 0.1 to about 10% by weight, or about 0.1 to about 2, or about 0.1 to about 1 weight % of at least one warming agent. In an example, the composition contains a warming agent in an amount of about 0.1% wt, about 0.2% wt, about 0.3% wt, about 0.4% wt, about 0.5% wt, about 0.6% wt, about 0.7% wt, about 0.8% wt, about 0.9% wt, or about 1.0% wt. In an example the composition contains about 0.5% by weight of at least one warming agent.

In an example, the composition may include one or more warming agents and one or more cooling agents to deliver a dual sensation to the consumer. The dual warming and cooling sensations may be simultaneously felt by the consumer, or the composition may be formulated to deliver the warming and cooling sensations at different time points. For example, the composition may be formulated to deliver an immediate cooling sensation, followed by a warming sensation after a certain amount of time, for example, 3, 4, 5, 6, 7, 8, 9, 10, 11, 12, 13, 14, 15, 16, 17, 18, 19, 20, 21, 22, 23, 24, or 25 minutes after application to the user's skin. Alternatively, the composition may be formulated to deliver an immediate warming sensation, followed by a cooling sensation after a certain amount of time, for example, 3, 4, 5, 6, 7, 8, 9, 10, 11, 12, 13, 14, 15, 16, 17, 18, 19, 20, 21, 22, 23, 24, or 25 minutes after application to the user's skin. One of the warming or the cooling sensations may pre-dominate the other. For example, the user may feel a strong cooling sensation and a milder warming sensation. Or, the user may feel a strong warming sensation and a milder cooling sensation. Likewise, the composition may be formulated to provide an equal warming and cooling sensation.

In an example, the composition may contain both camphor, as a warming agent, and menthol, as a cooling agent, to effectuate both a warming and cooling sensation. The camphor may be in an amount of about 3% weight to about 11% weight, or about 3.0% weight, about 3.5% weight, about 4.0% weight, about 4.5% weight, about 5.0% weight, about 5.5% weight, about 6.0% weight, about 6.5% weight, about 7.0% weight, about 7.5% weight, about 8.0% weight, about 8.5% weight, about 9.0% weight, about 9.5% weight, about 10.0% weight, about 10.5% weight, or about 11.0% weight. The menthol may be in an amount of about 1.25% weight to about 16% weight, for example about 1.25% weight, about 1.5% weight, about 1.75% weight, about 2.0% weight, about 2.5% weight, about 3.0% weight, about 3.5% weight, about 4.0% weight, about 4.5% weight, about 5.0% weight, about 5.5% weight, about 6.0% weight, about 6.5% weight, about 7.0% weight, about7.5% weight, about 8.0% weight, about 8.5% weight, about 9.0% weight, about 9.5% weight, about 10.0% weight, about 10.5% weight, about 11.0% weight, about 11.5% weight, about 12.0% weight, about 12.5% weight, about 13.0% weight, about 13.5% weight, about 14.0% weight, about 14.5% weight, about 15.0% weight, about 15.5% weight, or about 16.0% weight. The combination of camphor to menthol may be in a particular weight ratio, for example, from about 2:1 to about 4:1, or about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1 or about 4:1.

In one example, the composition may include an amount of about 4% by weight camphor and an amount of about 1.25% by weight menthol. In such example, the weight ratio of camphor to menthol may be about 3.2:1. Or, in reverse, the weight ratio of menthol to camphor may be about 1:3.2. Without being bound by theory, it is suggested that camphor and menthol form a eutectic system in which the ratio of menthol to camphor lowers the melting point of camphor and allows processing of the camphor and menthol within the temperature range (e.g., around 80° C.) of the other composition ingredients, like emollients and waxes. In other words, the higher the menthol to camphor ratio, the lower the melting point of the combination. Thus, the above-referenced weight ratio of camphor to menthol provides both 1) an acceptable or superior cooling and warming sensation to a user and 2) avoidance of the need to include additional viscosity increasing agents and/or emulsifiers in the composition to compensate for camphor's high melting point during processing.

In this example, it was further found that increasing the camphor from 4% by weight to 8% or 11% by weight and/or the menthol from 1.25% by weight to 2.5% or 5.0% by weight, did not contribute to any meaningful additional warming or cooling sensation to a user. For example, a composition combining 8% by weight camphor and 2.5% by weight menthol had no meaningful difference in consumer perception of warming and cooling sensation as compared to a composition combining 4% by weight camphor and 1.25% by weight menthol. Thus, the present example minimizes the use of camphor and menthol, while still providing acceptable or superior warming and cooling sensation to a user.

According to an example, the dual sensation composition described above may have an additional warming or cooling agent. For example, the composition may include menthol as a cooling agent and both camphor and VBE as warming agents. According to an example, VBE may be added in an amount of about 0.1% weight, or about 0.15% weight, or about 0.2% weight, or about 0.25% weight, or about 0.3% weight, or about 0.35% weight, or about 0.4% weight. In one example, the composition may include about 4.0% by weight camphor, about 1.25% by weight menthol and about 0.25% by weight VBE.

The composition may include one or more “thickeners,” or “viscosity increasing agents,” selected from, for example, ammonium acrylolydimethyltaurate/vinylpyrrolidone copolymer, sodium acrylolydimethyltaurate/vinylpyrrolidone copolymer, sodium polyacrylate, carbomer, and/or acrylates copolymer. According to an example, the composition may include about 0.1 to about 10, or about 0.05 to about 1.2, or about 0.6 weight % thickener. The amount and selection of the thickener allows for stabilization of the cream or gel cream while maintaining the cream or gel cream aesthetic and skin feel. The thickener(s) may be present in amounts ranging from about 0.01, 0.05, 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 to about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 5, 6, 7, 8, 9, 10 wt. % of the total composition.

According to an example, the viscosity increasing agent may be carbomer, for example as sold under tradename “Carbopol® Ultrez 10 Polymer” from Lubrizol Corporation. The carbomer may be in an amount of about 0.1% wt. According to an example, the viscosity increasing agent may be sodium polyacrylate, for example as sold under tradename “Cosmedia® SP” from the UL Prospector Corporation. The sodium polyacrylate may be in an amount of about 0.5 wt %. According to an example, the viscosity increasing agent may be a combination of carbomer and sodium polyacrylate.

As used herein, the term “emulsifier” refers to an ingredient that helps keep ingredients (such as oil and water) from separating in an emulsion. According to an example, an example of an emulsifier may include cetyl alcohol. According to an example, the composition may include about 0.1% to about 10%, or about 0.25 to about 5, or about 0.5 to 2.0 weight % emulsifier. In one example, the emulsifier may be about 1 wt. %. The emulsifier(s) may be present in amounts ranging from about 0.01, 0.05, 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 to about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 5, 6, 7, 8, 9, 10 wt. % of the total composition.

The compositions of the present invention are applied topically to human skin. Accordingly, the composition may further include cosmetically acceptable topical ingredients as known in the personal care art for use with cream formulations or gel-cream formulations of the oil-in-water emulsion type.

The composition may contain for example suitable gelling agents such as natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contain between about 0.1% and 5%, by weight, of such gelling agents.

The compositions of the present invention may further include any of a variety of additional cosmetically active agents. Examples of suitable additional active agents may include: anti-microbial, anti-fungal, and anti-bacterial agents, anti-inflammatory agents, additional external analgesics, sunscreens, photoprotectors, antioxidants, moisturizers, nutrients, vitamins, energy enhancers, hydration boosters, efficacy boosters, agents for skin conditioning, odor-control agents such as odor masking or pH-changing agents, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred examples, in an amount of 0.1% to 5% and in other preferred examples from 1% to 2%.

As used herein, a “skin conditioning agent,” or “skin conditioner” generally refers to an ingredient that enhances the appearance of dry or damaged skin by reducing flaking and/or restoring suppleness. For example, a skin conditioning agent may act as a lubricant on the skin surface to give the skin a soft and smooth appearance. Or, a skin conditioning agent may increase the water content of the top layer of the skin by drawing moisture from the surrounding air. In certain examples, the composition may include at least one skin conditioning agent. For example, in one example, the composition may include zingiber officinale (ginger) root extract, caprylyl glycol, 1,2-hexanediol and/or tropolone.

Compositions of the present invention may include a cosmetically effective amount of one or more skin conditioning agents such as those described above. The compositions preferably include, on an active basis, from about 0.1% to about 10% by weight of the skin conditioning agents, more preferably from about 0.5% to about 5% of skin conditioning agents, and most preferably from about 0.5% to about 2% of skin conditioning agents.

Compositions of the present invention may include a cosmetically effective amount of one or more anti-inflammatory compounds. Examples of suitable anti-inflammatory agents may include substituted resorcinols, (E)-3-(4-methylphenylsulfonyl)-2-propenenitrile (such as “Bay 11-7082,” commercially available from Sigma-Aldrich of St. Louis, Missouri), tetrahydrocurcuminoids (such as Tetrahydrocurcuminoid CG, available from Sabinsa Corporation of Piscataway, NJ), extracts and materials derived from the following: Phellodendron amurense Cortex Extract (PCE), Non-Denatured Soy (Glycine max), Feverfew (Tanacetum parthenium), Ginger (Zingiber officinale), Ginko (Ginkgo biloba), Madecassoside (Centella asiatica extract ingredient), Cotinus (Cotinus coggygria), Butterbur Extract (Petasites hybridus), Goji Berry (Lycium barbarum), Milk Thistle Extract (Silybum marianum), Honeysuckle (Lonicera japonica), Basalm of Peru (Myroxylon pereirae), Sage (Salvia officinalis), Cranberry Extract (Vaccinium oxycoccos), Amaranth Oil (Amaranthus cruentus), Pomegranate (Punica granatum), Yerbe Mate (Ilex paraguariensis Leaf Extract), White Lily Flower Extract (Lilium candidum), Olive Leaf Extract (Olea europaea), Phloretin (apple extract), Oat Flour (Aveena sativa), Lifenol (Hops: Humulus lupulus) Extract, Bugrane P (Ononis spinosa), Licochalcone (Licorice: Glycyrrhiza inflate extract ingredient), Symrelief (Bisabolol and Ginger extract), combinations of two or more thereof, and the like.

In certain examples the composition may include an anti-inflammatory compound in an amount from about 0.5% to about 2% by weight, and more preferably from about 0.5% to about 1.5%. In another example, the anti-inflammatory compound may include colloidal oat flour and/or ginger.

The pH of the composition may be adjusted using pH adjusting agents commonly used in the art. In one or more examples, the composition has a pH of from about 4.5 to about 5.5. In some examples, the pH of the composition ranges from about 3, 3.1, 3.2, 3.3, 3.4, 3.5, 3.6, 3.7, 3.8 or 3.9 to about 3.8, 3.9, 4, 4.1, 4.2, 4.3, 4.4, 4.5, 4.6, 4.7, 4.8, 4.9 or 5. In some examples, the pH of the composition ranges from about 3 to about 5, or about 3.5 to about 4.5. In other examples, the pH of the composition may range from about 6, 6.1, 6.2, 6.3, 6.4, 6.5, 6.6, 6.7, 6.8 or 6.9 to about 6.8, 6.9, 7, 7.1, 7.2, 7.3, 7.4, 7.5, 7.6, 7.7, 7.8, 7.9 or 8. In some examples, the pH of the composition ranges from about 6 to about 7.

In one example, the pH adjusting agent may be citric acid in an amount from about 0.1% to about 10% by weight, more preferably from about 0.5% to about 5%, and most preferably from about 0.5% to about 1% by weight. According to an example, the citric acid may be in an amount of about 0.6 wt. %.

“Emollients” may include compounds that help to maintain the soft, smooth, and pliable appearance of the skin (e.g., by remaining on the skin surface or in the stratum corneum to act as a lubricant). Examples of suitable emollients may include petrolatum, isopropyl palmitate, cetearyl olivate, sorbitan olivate, Euphorbia Cerifera (Candelila) wax, isododecane, hexyldecyl stearate and plant, nut, and vegetable oils and hydrogenated vegetable oils such as macadamia nut oil, rice bran oil, grape seed oil, palm oil, prim rose oil, hydrogenates peanut oil, and avocado oil. According to an example, the emollient may be isopropyl palmitate, for example as sold under tradename “Radia 7732” from the Oleon Corporation. The isopropyl palmitate may be used in an amount of 3 wt. %. According to another example, the emollient may be a combination of cetearyl olivate and sorbitan olivate, for example, as sold under the tradename “Olivem® 1000” by the Hallstar BPC Corporation. The cetearyl olivate and sorbitan olivate may be used in a total amount of 0.5 wt. %.

According to an example, the emollient may include a combination of isopropyl palmitate, cetearyl olivate and sorbitan olivate. In certain examples the composition may include combined emollients in an amount from about 0.1% to about 15% by weight, and more preferably from about 1% to about 5%, or about 2.5% to about 5.0%. According to one example, the combined emollients are about 3.5 wt. %. In this example each emollient may be present from about 0.5 to about 3.0% by weight. In this example the isopropyl palmitate may be present at about 3.0% by weight and the cetearyl olivate: sorbitan olivate may be present at about 0.5% by weight. The emollient(s) may be present in amounts ranging from about 0.01, 0.05, 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 to about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 5, 6, 7, 8, 9, 10 wt. % of the total composition.

As used herein, “oils” means organic, hydrophobic compounds that are liquid at 25° C. excluding silicone-based materials. Examples of organic oils include various hydrocarbons (straight or branched chain alkanes or alkenes, ketone, diketone, primary or secondary alcohols, aldehydes, sterol esters, alkanoic acids, turpenes, monoesters), such as those having a carbon chain length ranging from C6-C38, such as C6-C18.

Specific non-limiting examples of oils include without limitation natural oils such as castor oil, mineral oil, trigylcerides, esters of an alcohol (glycerol or other than glycerol including diesters or other branched esters) and a fatty acid or fatty alcohol, and various natural waxes including shea (Butyrospermum parkii) butter, lotus wax; beeswax, insect waxes, sperm whale oil, lanolin, vegetable waxes such as canauba wax, jojoba oil, candelilla wax; mineral waxes such as paraffin wax; and synthetic waxes such as cetyl palmitate, lauryl palmitate, cetostearyl stearate, and polyethylene wax.

Oils may include for example esters such as, isopropyl myristate, isononyl isonanoate (such as WICKENOL 151 available from Alzo Inc. of Sayreville, NJ), C12-C15 alkyl benzoates (such as FINSOLV TN from Innospec Active Chemicals), caprylic/capric triglycerides, pentaerythritol tetraoctanoate, mineral oil, dipropylene glycol dibenzoate, PPG-15 stearyl ether benzoate, PPG-2-Myristyl Ether Propionate, ethyl methicone, and diethylhexylcyclohexane. Further examples of oils include functional oils such as vitamin E acetate.

What is meant by a “humectant” is a compound intended to increase the water content of the top layers of skin (e.g., hygroscopic compounds). Examples of suitable humectants include, but are not limited to, glycerin, sorbitol or trehalose (e.g., α,α-trehalose, β,β-trehalose, α,β-trehalose) or a salt or ester thereof (e.g., trehalose 6-phosphate). In one example, the humectant is glycerin (also spelled “glycerine” interchangeably). The humectant(s) may be present in amounts ranging from about 0.01, 0.05, 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 to about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 5, 6, 7, 8, 9, 10 wt. % of the total composition.

In certain examples the composition may include a humectant in an amount from about 1% to about 20% by weight, and more preferably from about 1.0% to about 5.0% by weight. According to one example, the humectant is about 3.0% by weight. In an example, the humectant may be glycerine in an amount of about 3.0 wt. %.

As used herein, a “preservative” refers to an ingredient that may prevent or retard bacterial growth and thus protect a cosmetic product from spoilage. Suitable preservatives include, for example, phenoxyethanol, caprylyl glycol, 1,2-hexanediol, tropolone and/or chlorphenesin and are present in the composition in an amount, based upon the total weight of the composition, from about 0 to about 5 percent or from about 0.05 percent to about 2 percent. According to one example, the preservative may be phenoxyethanol present in an amount of about 0.50 wt. %.

Any of a variety of commercially available pearlescent or opacifying agents are suitable for use in the composition. Examples of suitable pearlescent or opacifying agents include, but are not limited to, mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO—(JO)ₐ—H, wherein J is an alkylene group having from about 2 to about 3 carbon atoms; and a is 2 or 3; fatty alcohols containing from about 16 to about 22 carbon atoms; fatty esters of the formula: KCOOCH₂L, wherein K and L independently contain from about 15 to about 21 carbon atoms; inorganic solids insoluble in the composition, and mixtures thereof.

Any fragrance compositions suitable for use on skin may be used in accordance with the present invention. A fragrance may be in the amount of 0.1% to 2% by weight.

According to an example, the composition may contain water, for example purified water, to serve as a vehicle. Water may be added from about 50% to about 95% by weight of the composition, or from about 75% to about 90% by weight of the composition, or about 80% to about 90% by weight. Water may be added to achieve quantum satis (“q.s.” or “Q.S.”) in a formulation.

A variety of other materials may optionally be present in the compositions of the present invention. In certain preferred examples, the composition includes one or more topical ingredients selected from the group consisting of: surfactants, chelating agents, additional emollients, humectants, conditioners, preservatives, opacifiers, fragrances and the like.

### Product Form and Packaging

A variety of product forms and packaging may be suitable for any of the compositions described herein. As noted earlier, a “product” is optionally in finished packaged form. In one example, the package is a container such as a plastic, metal or glass tube or jar containing the composition. In one or more examples, the composition may be squeezed or pumped out of the container. The product may further contain additional packaging such as a plastic or cardboard box for storing such container. In one or more examples, the product includes a composition of the invention and contains instructions directing the user to apply the composition to the skin. In one example the packaging is a tube with at least one orifice to dispense the product. The top of the tube may be fitted with a rollerball or up to three rollerballs to facilitate massaging of the skin area while applying the product.

Any of the compositions may be in the form of emulsions, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. Reference in the instant application is made to two types of viscosity measurements: A) “rheometer viscosity,” which is measured as a steady-state value at an applied shear rate of 20 s⁻¹ in a rheometer, at 25° C., and B) “Brookfield viscosity,” which is measured at 5 or 10 RPM after one minute at 25° C. in 4 oz jar using spindle RV #4 or RV #5. Unless otherwise specified, the viscosity referenced is the rheometer viscosity.

In one or more examples, the compositions described herein may be in the form of a lotion, gel, cream or gel-cream product. Lotion, gel, cream and gel-cream products may advantageously have the properties to allow for the compositions to be flowable and applied onto skin. Such products are generally thin enough to allow the volatile emollient to evaporate and provide the cooling effect. If too thick, as with an ointment, occlusive or patch, the user may not perceive a cooling affect.

In one or more examples, the compositions described herein are in the form of a lotion or a cream. As used herein, the term “lotion” may mean a predominantly water-containing topical preparation of light texture and fresh watery sensation. The term “cream” may mean a predominantly water-containing topical preparation of rich texture. Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 1% to about 5%) of an emulsifier(s).

Single emulsion skin care preparations, such as lotions and creams, of the oil-in-water type, and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel. As used herein, the term “gel” means a predominantly water-containing topical preparation of rich texture containing dispersing polymer and exhibiting yield value of about 0.1 Pa or more. The gel may contain a gelling agent. Such gels typically contain between about 0.1% and 5%, by weight, of such gelling agents.

According to a further example, compositions of this invention may be formulated as a gel-cream. As used herein, the term “gel-cream” means a predominantly water-containing topical preparation of rich texture that has some user aesthetics of both a cream and a gel, as defined above.

A gel-cream may contain from about 1% to about 20% (e.g., from about 1% to about 5%) of an emollient(s) and from about 45% to about 95% (e.g., from about 70% to about 90%) of water. A gel-cream may contain a dispersing polymer and exhibit a yield value of about 0.1 Pa or more. A gel-cream may contain one or more gelling agents, for example, between about 0.1% and 5% by weight of such gelling agents.

The compositions of the present invention may have a viscosity range of 16,000 cps to 140,000 cps when tested at 25° C.±1° C.: using a Brookfield LV Helipath TE viscometer set at 3 RPM for 1 minute. The composition may have a viscosity from about 20,000 to about 85,000 cps, or from about 30,000 to about 60,000 cps. Additionally the composition may have a viscosity from about 30,000 cps to about 60,000 cps after storage at 3 months and 40° C., and from about 40,000 cps to about 85,000 cps after 3 months of cycling in Freeze/Thaw conditions.

The compositions of the present invention can also be formulated into a solid formulation (e.g., wipe). The composition of the present invention can also be combined with and/or impregnated on to a solid, semi-solid, or dissolvable substrate (e.g., a wipe, mask, pad, glove, or strip).

The compositions described herein can be provided to the consumer in a container, e.g., a bottle, tube, etc. Individual packets enclosing measured portions of the composition may also be used. To dispense the composition from a bottle, a pump, squeezable valve, or a removable screw cap may be used.

### Methods

The present invention further includes a method of relieving pain by topically applying the composition of the invention to the skin in areas in need of pain relief. The method includes, for example, topically applying the composition to skin in a location of the body in need of pain relief. Such topical application may be to any skin in need of treatment on the body, for example skin of the face, neck, chest, back, hips, arms, axilla, hands, feet and/or legs.

The present invention includes a method of relieving pain by topically applying the composition of the invention to the skin in areas in need of pain relief, where the user or subject has sensitive skin. The method includes, for example, topically applying the composition to sensitive skin in a location of the body in need of pain relief. Such topical application may be to any skin in need of treatment on the body, for example skin of the face, neck, chest, back, hips, arms, axilla, hands, feet and/or legs.

The compositions may be applied to the skin as needed, or otherwise how often the user desires. In one or more examples, the composition is applied once or twice per day.

Any suitable method of applying the composition to the skin in need may be used. For example, the composition may be applied directly from a package to the skin in need, by hand to the skin in need, using an applicator to the skin in need, or may be transferred from a substrate such as a wipe or mask, or a combination of two or more thereof. In other examples, the composition may be applied via a dropper, tube, roller, pump, and patch or added to a bath or otherwise to water to be applied to the skin, and the like. The composition may be applied in a variety of manners/forms, including, without limitation, as a leave-on cream, a rinse-off cream, and/or as part of a patch.

The composition, and formulations and products containing such composition, may be prepared using methodology that is well known by an artisan of ordinary skill. The composition may be used on a routine basis and is substantially free of skin irritants.

### EXAMPLES

The following examples set forth below further illustrate examples of the present invention. All amounts are given in weight percent. The present invention is not limited to the examples contained therein.

### Example 1: Warming Topical Cream with Lidocaine

A topical cream composition according to an example of the invention was made having the following ingredients, as shown in Table 1.

TABLE 1
Ingredient Listing for Example 1

| Ingredient | Primary Function | Weight % |
|---|---|---|
| Purified Water | Vehicle | 86.65 |
| Carbomer¹ | Viscosity Increasing Agent | 0.10 |
| Lidocaine USP | Topical Analgesic | 4.00 |
| Glycerine | Humectant | 3.00 |
| Sodium Polyacrylate² | Viscosity Increasing Agent | 0.50 |
| Isopropyl Palmitate³ | Emollient | 3.00 |
| Cetearyl Olivate; Sorbitan Olivate⁴ | Emollient | 0.50 |
| Cetyl Alcohol⁵ | Emulsifier | 1.00 |
| Phenoxyethanol⁶ | Preservative | 0.50 |
| Vanilla Butyl Ether⁷ | Warming Agent | 0.50 |
| Fragrance | Fragrance | 0.25 |

¹Commercially available as Carbopol ® Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Phenoxetol NF from the Clariant Corporation
⁷Commercially available as HOTACT ® from the Takasago Corporation

The following mixing procedure may be used to prepare Example 1:
a) The purified water is added to a suitable vessel and the lidocaine USP is added while mixing and mixed until dissolved
b) The carbomer is added while mixing slowly until all clumps are dissolved
c) The mixture is heated until 75-80° C.
d) The sodium polyacrylate is then added slowly while mixing at 75-80° C. until uniform
e) The oil phase ingredients are then added while mixing (approximately 50% of the glycerin, isopropyl palmitate, cetearyl olivate; sorbitan olivate, and cetyl alcohol)
f) The mixture is mixed for 10 minutes and then homogenized for 3 minutes at 5000 RPM
g) The mixture is cooled to 25° C. and additional 50% of glycerin are added and mixed
h) The remaining ingredients are added and mixed until homogeneous
i) The batch is brought to volume with water to 95-100% of theoretical batch weight.

### Example 2: Cooling Topical Cream with Lidocaine

A topical cream composition according to an example of the invention was made having the following ingredients, as shown in Table 2.

TABLE 2
Ingredient Listing for Example 2

| Ingredient | Primary Function | Weight % |
|---|---|---|
| Purified Water | Vehicle | 85.40 |
| Carbomer¹ | Viscosity Increasing Agent | 0.10 |
| Lidocaine USP | Topical Analgesic | 4.00 |
| Glycerine | Humectant | 3.00 |
| Sodium Polyacrylate² | Viscosity Increasing Agent | 0.50 |
| Isopropyl Palmitate³ | Emollient | 3.00 |
| Cetearyl Olivate; Sorbitan Olivate⁴ | Emollient | 0.50 |
| Cetyl Alcohol⁵ | Emulsifier | 1.00 |
| Phenoxyethanol⁶ | Preservative | 0.50 |
| Menthol Laevo Pellets (USP) | Cooling Agent | 1.00 |
| Fragrance | Fragrance | 1.00 |

¹Commercially available as Carbopol ® Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Phenoxetol NF from the Clariant Corporation

The following mixing procedure may be used to prepare Example 2:
j) The purified water is added to a suitable vessel and the lidocaine USP is added while mixing and mixed until dissolved
k) The carbomer is added while mixing slowly until all clumps are dissolved
l) The mixture is heated until 75-80° C.
m) The sodium polyacrylate is then added slowly while mixing at 75-80° C. until uniform
n) The oil phase ingredients are then added while mixing (approximately 50% of the glycerin, isopropyl palmitate, cetearyl olivate; sorbitan olivate, and cetyl alcohol)
o) The mixture is mixed for 10 minutes and then homogenized for 3 minutes at 5000 RPM
p) The mixture is cooled to 25° C. and additional 50% of glycerin are added and mixed
q) The remaining ingredients are added and mixed until homogeneous.
r) The batch is brought to volume with water to 95-100% of theoretical batch weight.

### Example 3: Topical Cream with Lidocaine without a Sensate

A topical cream composition according to an example of the invention was made having the following ingredients, as shown in Table 3.

TABLE 3
Ingredient Listing for Example 3

| Ingredient | Primary Function | Weight % |
|---|---|---|
| Purified Water | Vehicle | 87.40 |
| Carbomer¹ | Viscosity Increasing Agent | 0.10 |
| Lidocaine USP | Topical Analgesic | 4.00 |
| Glycerine | Humectant | 3.00 |
| Sodium Polyacrylate² | Viscosity Increasing Agent | 0.50 |
| Isopropyl Palmitate³ | Emollient | 3.00 |
| Cetearyl Olivate; Sorbitan Olivate⁴ | Emollient | 0.50 |
| Cetyl Alcohol⁵ | Emulsifier | 1.00 |
| Phenoxyethanol⁶ | Preservative | 0.50 |

¹Commercially available as Carbopol ® Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Phenoxetol NF from the Clariant Corporation

The following mixing procedure may be used to prepare Example 3:
s) The purified water is added to a suitable vessel and the lidocaine USP are added while mixing and mixed until dissolved
t) The carbomer is added while mixing slowly until all clumps are dissolved
u) The mixture is heated until 75-80° C.
v) The sodium polyacrylate is then added slowly while mixing at 75-80° C. until uniform
w) The oil phase ingredients are then added while mixing (approximately 50% of the glycerin, isopropyl palmitate, cetearyl olivate; sorbitan olivate, and cetyl alcohol)
x) The mixture is mixed for 10 minutes and then homogenized for 3 minutes at 5000 RPM
y) The mixture is cooled to 25° C. and additional 50% of glycerin are added and mixed
z) The remaining ingredients are added and mixed until homogeneous
aa) The batch is brought to volume with water to 95-100% of theoretical batch weight.

### Example 4: Dual Sensation Topical Cream with Camphor and Menthol

A topical cream composition according to an example of the invention was made having the following ingredients, as shown in Table 4.

TABLE 4
Ingredient Listing for Example 4

| Ingredient | Primary Function | Weight % |
|---|---|---|
| Purified Water | Vehicle | 84.90 |
| Carbomer¹ | Viscosity Increasing Agent | 0.10 |
| Camphor | Topical Analgesic/Warming | 4.00 |
| | Agent | |
| Glycerin | Humectant | 3.00 |
| Sodium Polyacrylate² | Viscosity Increasing Agent | 0.50 |
| Isopropyl Palmitate³ | Emollient | 3.00 |
| Cetearyl Olivate; Sorbitan Olivate⁴ | Emollient | 0.50 |
| Cetyl Alcohol⁵ | Emulsifier | 1.00 |
| Phenoxyethanol⁶ | Preservative | 0.50 |
| Menthol | Topical Analgesic/Cooling | 1.25 |
| | Agent | |
| Vanilla Butyl Ether⁷ | Warming Agent | 0.25 |
| Fragrance | Fragrance | 1.00 |

¹Commercially available as Carbopol ® Ultrez 10 Polymer from the Lubrizol Corporation
²Commercially available as Cosmedia ® SP from the UL Prospector Corporation
³Commercially available as Hallstar IPP-NF from the Hallstar BPC Corporation
⁴Commercially available as Olivem 1000 from the Hallstar BPC Corporation
⁵Commercially available as CO-1695 from the Hallstar BPC Corporation
⁶Commercially available as Phenoxetol NF from the Clariant Corporation
⁷Commercially available as HOTACT ® from the Takasago Corporation

The following mixing procedure may be used to prepare Example 3:
a) The purified water is added to a suitable vessel
b) The carbomer is added while mixing slowly
c) The mixture is heated until 80° C. and mixed until dissolved and cooled to 70-75° C.
d) The glycerin and sodium polyacrylate are then added slowly while mixing at 70-75° C. until uniform
e) The camphor and menthol are added and mixed at 80° C. until melted
f) The cetearyl olivate; sorbitan olivate, and cetyl alcohol are then added while maintaining the mixture at 80° C.
g) The isopropyl palmitate is then added and mixed at 80° C. for 3 minutes
h) The heat is turned off and once the batch is below 35° C., the phenoxyethanol, vanilla butyl ether and fragrance are then added and mixed
i) The batch is brought to volume with water to 95-100% of theoretical batch weight.

### Human Use Test Study of Examples 1-3

### Study Protocol and Objective

A home use test (abbreviated as “HUT”) was conducted where between 260 and 280 consumers (also referred to as “subjects,” “patients” or “users”) self-administered the formulas in Examples 1, 2 and 3 (also referred to in this example as “formula(s)” or “product(s)”) as needed for a period of three days. More specifically, 270 consumers used the formula in Example 1, 277 consumers used the formula in Example 2, and 280 consumers used the formula in Example 3. These consumers were between 45 and 64 years old who regularly or occasionally experience minor aches and pains of the muscles or joints, and must regularly or occasionally experience minor aches and pains of muscles/joints in the past month and/or experience arthritis.

The consumers provided feedback via an online survey at three time points: 1) after initial use, 2) after 15 minutes, and 3) a final assessment after use.

Example 1 may be referred to as a “warming cream” incorporating, for example, a warming agent such as vanilla butyl ether and Example 2 may be referred to as a “cooling cream” incorporating a suitable calming cooling agent, for example, menthol and Example 3 may be referred to as a “simply cream” without a warming or cooling sensate.

The objective of the human use study was to generate support for potential consumer perceptual claims centered around a consumers' experience with the product. For example, an objective was to understand the consumers' experience with regard to warming or cooling, ease of use, feeling of stickiness or greasiness, absorption and feel of absorption on the skin.

A “test statement” may be a question that is answered or a statement that is agreed or disagreed with at least once by the consumer during the duration of the HUT. According to an example, the following test statements may relate to the consumers experience with regard to product absorption: is fast absorbing, instantly absorbs into the skin, is non-sticky, or non-greasy, and/or doesn't leave a residue. According to an example the test statement of easy to apply and mess-free application may relate to the consumers experience with application. The test statements of instantly cools, feels like it is working right away and/or instantly numbs may indicate pain relief.

### Summary of the Results

The top line data is presented in Tables 5 and 6, and shows the reactions as described by consumer, collated as a percentage of users in agreement with the test statement. Total results, as well as results for each of Example 1 and Example 2 are shown.

TABLE 5
Human Use Study Results-Primary Attributes

| | | Percentage (%) of Consumers | | |
|---|---|---|---|---|
| | | in Agreement | | |
| | | Example | Example | Example |
| | Time of | 1 | 2 | 3 |
| Test Statement | statement | (n = 270) | (n = 277) | (n = 280) |
| is easy to apply | After immediate | 98 | 97 | 98 |
| | use | | | |
| Mess-free | After immediate | 94 | 94 | 96 |
| application | use | | | |
| Is instantly cooling | After immediate | | 82 | |
| | use | | | |
| Provides a cooling | After 15 minutes | | 89 | |
| sensation after 15 | | | | |
| minutes | | | | |
| Is instantly | After immediate | 64 | | |
| warming | use | | | |
| Provides a long- | After 15 minutes | 60 | | |
| lasting warming | | | | |
| is non-sticky | After 15 minutes | 95 | 97 | 98 |
| is non-greasy | After 15 minutes | 94 | 96 | 96 |
| Doesn't leave a | After 15 minutes | 93 | 92 | 94 |
| residue | | | | |
| Rapidly absorbs | After 15 minutes | 93 | 95 | 93 |

* Not Applicable: Formula was fragrance free.

Over 90% of consumers found both Examples 1, 2 and 3 to be easy to apply and have a mess free application after immediate use. Over 90% of consumers also found Examples 1, 2 and 3 to be non-sticky, non greasy, not to leave a residue and rapidly absorbed after 15 minutes. Greater than 80% of consumers also agreed that the cooling cream was instantly cooling after immediate use and provided a cooling sensation after 15 minutes. Greater than 60% of consumers also agreed that the warming cream provided a warming sensation after 15 minutes and long-lasting warming.

TABLE 6
Secondary Attributes and Unmet Needs

| | Percentage (%) of Consumers in | | |
|---|---|---|---|
| | Agreement | | |
| | Example 1 | Example 2 | Example 3 |
| Test Statement | (n = 270) | (n = 277) | (n = 280) |
| Doesn't make my skin feel dry/tight | 97 | 96 | 96 |
| Lightweight | 92 | 96 | 95 |
| Leaves my skin feeling soothed | 83 | 92 | 82 |
| Leaves skin feeling moisturized | 80 | 84 | 79 |
| Provides relaxing comfort | 79 | 86 | 75 |
| I feel using this product doesn't | 97 | 97 | 96 |
| interrupt my daily routine | | | |
| I didn't feel self-conscious wearing | 95 | 96 | 95 |
| this product when going about my day | | | |
| I felt I could use this product | 92 | 94 | 94 |
| discretely | | | |
| Doesn't rub off on clothes and/or | 91 | 85 | 92 |
| sheets | | | |
| I felt this product fit seamlessly into | 92 | 91 | 89 |
| my lifestyle | | | |
| I feel more in control of my pain | 69 | 73 | 66 |

Greater than 60% of consumers also agreed that Examples 1, 2 and 3 didn't make the consumer's skin feel dry-tight, were lightweight, left skin feeling soothed and moisturized, provided relaxing comfort, felt like the product didn't interrupt daily routine, didn't feel self-conscious wearing the product when going about their day, felt like they could use the product discreetly, didn't rub off on clothes and/or sheets, felt like the product fit into their lifestyle and felt more in control of their pain. Overall, Example 2 (Cooling cream) left the skin feeling more moisturized and provided more relaxing comfort than Examples 1 and 3.

The areas of application on the body were also recorded to determine areas of preference. These are shown in Table 7.

TABLE 7
Applications to Areas of the Body

| | Preferred application site by | | |
|---|---|---|---|
| | Percentage (%) of Consumers | | |
| | Example 1 | Example 2 | Example 3 |
| Area of Body | (n = 270) | (n = 277) | (n = 280) |
| Back | 46 | 50 | 40 |
| Shoulders | 42 | 38 | 28 |
| Legs | 38 | 34 | 36 |
| Neck | 32 | 33 | 27 |
| Hands/Fingers/Wrists | 38 | 32 | 25 |
| Arms | 17 | 18 | 21 |
| Other | 18 | 15 | 18 |

The results show that the back, shoulders and legs were the most preferred locations for application, while Example 1 and Example 2 had higher application preference on the shoulders.

Subsets of consumers from the study in Table 7 were additionally given a test statement in terms of agreement that the composition “provides a warming sensation” for Example 1 (Warming Topical Cream) and “provides a cooling sensation” for Example 2 (Cooling Topical Cream). The results are show in Table 8, with the “n,” or number of consumers who applied the compositions to those specific areas of the body. The data is compiled based on the percentage of consumers who “agree” or “strongly agree” with the test statement.

TABLE 8
Warming or Cooling Sensation on Areas of the Body

| | Percent of Agreement using site (%) | |
|---|---|---|
| | of Consumers | |
| Area of Body | Example 1 (n) | Example 2 (n) |
| Back | 85 (n = 14) | 90 (n = 28) |
| Shoulders | 43 (n = 12) | 67 (n = 11) |
| Legs | 55 (n = 13) | 93 (n = 10) |
| Neck | 100 (n = 3) | 100 (n = 10) |
| Hands/Fingers/Wrists | 74 (n = 11) | 73 (n = 4) |
| Arms | 37 (n = 8) | 100 (n = 3) |

The results demonstrate definitive agreement for both compositions that a warming or cooling sensation is provided when applied to the back, legs, neck and/or hands/fingers/wrists. Of note, 100% of consumers agreed that Examples 1 and 2 provided a warming or cooling sensation, respectively, when applied to the neck. Interestingly, while 100% of consumers agreed that Example 2 provided a cooling sensation on the arms, only 37% of consumers agreed that Example 1 provided a warming sensation on the arms. Similar results were seen on the legs.

### Human Use Test Study of Example 4

### Study protocol and Objective

A home use test (abbreviated as “HUT”) was conducted where 109 consumers (also referred to as “subjects,” “patients” or “users”) self-administered the formula in Example 4 (also referred to in this example as “formula(s)” or “product(s)”) as needed for a period of seven days. These consumers were between 26 and 75 years old who regularly or occasionally experience minor aches and pains of the muscles or joints, and must regularly or occasionally experience minor aches and pains of muscles/joints in the past month and/or experience arthritis.

The consumers provided feedback via an online survey at three time points: 1) after initial use, 2) after a delay of fifteen (15)n exit survey after use.

The objective of the human use study was to generate support for potential consumer perceptual claims centered around a consumers' experience with the product. For example, an objective was to understand the consumers' experience with regard to warming or cooling, ease of use, feeling of stickiness or greasiness, absorption and feel of absorption on the skin.

A “test statement” may be a question that is answered or a statement that is agreed or disagreed with at least once by the consumer during the duration of the HUT. According to an example, the following test statements may relate to the consumers experience with regard to product absorption: is fast absorbing, instantly absorbs into the skin, is non-sticky, or non-greasy, and/or doesn't leave a residue. According to an example the test statement of easy to apply and mess-free application may relate to the consumers experience with application. The test statements of instantly cools, feels like it is working right away and/or instantly numbs may indicate pain relief.

### Summary of the Results

The top line data is presented in Table 9, and shows the reactions as described by consumer, collated as a percentage of users in agreement with the test statement. Results are shown for each of the time points: 1) after initial use, 2) after a delay of fifteen (15) minutes, and 3) an exit survey after use.

TABLE 9
Human Use Study Results for Example 4

| | Percentage (%) of Consumers in | | |
|---|---|---|---|
| | Agreement | | |
| Test Statement | Initial Use | Delayed | Exit Survey |
| “The Product . . . ” | Survey #1 | Survey #2 | #3 |
| Felt cooling on contact | 90 | | |
| Felt cooling | 90 | 90 | |
| Felt warming | 47 | 63 | |
| Provides a dual sensation | 64 | 67 | |
| Goes on clean | 82 | | 86 |
| Dries quickly | 74 | | 86 |
| Rapidly absorbs | 75 | | 87 |
| Is mess free | 76 | 82 | 84 |
| Is non-greasy | | 87 | 87 |
| Has no residue | | 85 | 86 |
| Doesn't stick to clothes | | | 88 |
| and sheets | | | |

Ninety (90) percent of consumers found Example 4 to be cooling on contact and cooling during the initial use survey and the delayed survey. Only 47% of consumers found Example 4 to feel warming at the initial use time point, whereas 63% of consumers found Example 4 to feel warming at the delayed time point. This may indicate that Example 4 has a delayed warming sensation. Greater than 85% of consumers found Example 4 to go on clean, dry quickly, rapidly absorb, be mess free, be non-greasy, leave no residue and not stick to sheets during the exit survey.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

We claim:

1. A topical composition comprising:
a topical analgesic,
a viscosity increasing agent in an amount of from about 0.05 to about 1.2 wt %,
a humectant in an amount of from about 1.0 to about 5.0 wt %,
an emollient in an amount of from about 2.5 to about 5.0 wt %, and
an emulsifier in an amount of from about 0.5 to about 2.0 wt %,
wherein the topical composition is substantially free of volatile alcohol.

2. The topical composition of claim 1, wherein the topical analgesic is in an amount of from about 0.5% to about 10% by weight.

3. The topical composition of claim 1, wherein the topical analgesic comprises one or more of lidocaine, camphor, menthol or benzocaine.

4. The topical composition of claim 3, wherein the topical analgesic comprises lidocaine.

5. The topical composition of claim 4, wherein the lidocaine is in an amount of about 4% by weight.

6. The topical composition of claim 3, wherein the topical analgesic comprises camphor and menthol.

7. The topical composition of claim 6, wherein the camphor is in an amount of about 4% by weight and the menthol is in an amount of about 1.25% by weight.

8. The topical composition of claim 1, further comprising a cooling agent.

9. The topical composition of claim 8, wherein the cooling agent comprises menthol or menthol lactate.

10. The topical composition of claim 1, further comprising a warming agent.

11. The topical composition of claim 10, wherein the warming agent comprises camphor, vanilla butyl ether or other vanilloid.

12. The topical composition of claim 11, wherein the warming agent comprises vanilla butyl ether or other vanilloid in an amount of about 0.25 to about 0.5% by weight.

13. The topical composition of claim 1, further comprising a warming agent and a cooling agent.

14. The topical composition of claim 13, wherein the warming agent comprises one or more of camphor, vanilla butyl ether or other vanilloid.

15. The topical composition of claim 13, wherein the cooling agent comprises menthol or menthol lactate.

16. The topical composition of claim 13, wherein the warming agent comprises camphor and the cooling agent comprises menthol.

17. The topical composition of claim 16, wherein the camphor and the menthol are in a weight ratio of 3.2:1.

18. The topical composition of claim 16, wherein the warming agent further comprises vanilla butyl ether.

19. The topical composition of claim 1, wherein the topical composition is free of a cooling agent or a warming agent.

20. The topical composition of claim 1, wherein the topical composition is free of lidocaine.

21. The topical composition of claim 1, wherein the topical composition is free of menthol.

22. The topical composition of claim 1, wherein the topical composition is free of camphor.

23. The topical composition of claim 1, wherein the viscosity increasing agent is in an amount of about 0.6% by weight.

24. The topical composition of claim 1, wherein the humectant is in an amount of about 3.0% by weight.

25. The topical composition of claim 1, wherein the at least one emollient is in an amount of about 3.5% by weight.

26. The topical composition of claim 1, wherein the emulsifier is in an amount of about 1.0% by weight.

27. A method of relieving pain in a user with sensitive skin, comprising applying the topical composition of claim 1 to an exterior skin portion of the user's body in need of pain treatment.

28. The method of claim 27, wherein the exterior skin portion of the user's body is at least one of the user's neck, back. legs, hands, fingers and wrists.
